(19) United States
(12) Patent Application Publication
(10) Pub. No.: US 2024/0134453 A1
(43) Pub. Date: Apr. 25, 2024

(54) DATA-EFFICIENT TRANSFER LEARNING FOR NEURAL DECODING APPLICATIONS

(71) Applicant: PRECISION NEUROSCIENCE CORPORATION, New York, NY (US)

(72) Inventors: Benjamin I. RAPOPORT, New York, NY (US); Craig H. MERMEL, New York, NY (US); Daniel TRIETSCH, New York, NY (US); Elton HO, New York, NY (US); Kazutaka TAKAHASHI, New York, NY (US)

(73) Assignee: PRECISION NEUROSCIENCE CORPORATION, New York, NY (US)

(21) Appl. No.: 18/493,323

(22) Filed: Oct. 23, 2023

Related U.S. Application Data
(60) Provisional application No. 63/418,657, filed on Oct. 24, 2022.

(51) Int. Cl. G06F 3/01; A61B 5/293; G06N 20/00

(52) U.S. Cl. CPC G06F 3/015; A61B 5/293; G06N 20/00; A61B 2560/0223; A61B 2562/046

(57) ABSTRACT

A systems and methods for calibrating a neural device using transfer learning techniques. The methods can include aggregating calibration data across a user population to define a global dataset, identifying similar data segments across the global dataset to define a task-independent training dataset, training a feature extraction model based on the task-independent training dataset to define a trained, task-independent feature extraction model, receiving the calibration data from a user calibrating the neural device, and calibrating a user-specific feature extraction model using the trained, task-independent feature extraction model and the calibration data.

### PRIORITY

The present application claims priority to U.S. Provisional Patent Application No. 63/418,657, titled DATA-EFFICIENT TRANSFER LEARNING FOR NEURAL DECODING APPLICATIONS, filed Oct. 24, 2022, which is hereby incorporated by reference herein in its entirety.

### BACKGROUND

Brain—computer interfaces have shown promise as systems for restoring, replacing, and augmenting lost or impaired neurological function in a variety of contexts, including paralysis from stroke and spinal cord injury, blindness, and some forms of cognitive impairment. Multiple innovations over the past several decades have contributed to the potential of these neural interfaces, including advances in the areas of applied neuroscience and multichannel electrophysiology, mathematical and computational approaches to neural decoding, power-efficient custom electronics and the development of application-specific integrated circuits, as well as materials science and device packaging. Nevertheless, the practical impact of such systems remains limited, with only a small number of patients worldwide having received highly customized interfaces through clinical trials.

High bandwidth brain-computer interfaces are being developed to enable the bidirectional communication between the nervous system and external computer systems in order to assist, augment, or replace neurological function lost to disease or injury. A necessary capability of any brain-computer interface is the ability to accurately decode electrophysiologic signals recorded from individual neurons, or populations of neurons, and correlate such activity with one or more sensory stimuli or intended motor response. For example, such a system may record activity from the primary motor cortex in an animal or a paralyzed human patient and attempt to predict the actual or intended movement in a specific body part; or the system may record activity from the visual cortex and attempt to predict both the location and nature of the stimuli present in the patient's visual field.

While increasingly accurate, real-time decoding systems have been described in the literature, a key limitation of existing systems is the need to calibrate the neural decoding algorithms for the neural devices on a patient-by-patient basis. This can be both a time-consuming process (because it requires that a significant amount of training data be obtained from the patient in order to calibrate the neural decoding algorithm) and results in decoding algorithms that lack robustness because they are tuned specifically to a single patient based solely on data generated from that single patient. Therefore, it would be beneficial to implement transfer learning techniques in neural systems so that the decoding algorithms can be trained using less active training data from each individual patient. Further, the application of transfer learning techniques to neural decoding applications would provide the ability to build neural decoding algorithms that are more robust to real-world variation. Still further, transfer learning techniques would also allow for the ability to train decoding algorithms that can perform tasks that would be infeasible to train using only a single individual's training data. Still further, implementing transfer learning techniques for neural decoding applications would also be beneficial because being able to use previously acquired patient data would improve the calibration speed and performance robustness for new or future patients.

Furthermore, brain-penetrating microelectrode arrays have facilitated high-spatial-resolution recordings for brain—computer interfaces, but at the cost of invasiveness and tissue damage that scale with the number of implanted electrodes. In some applications, softer electrodes have been used in brain-penetrating microelectrode arrays; however, it is not yet clear whether such approaches offer a substantially different tradeoff as compared to conventional brain-penetrating electrodes. For this reason, non-penetrating cortical surface microelectrodes represent a potentially attractive alternative and form the basis of the system described here. In practice, electrocorticography (ECoG) has already facilitated capture of high quality signals for effective use in brain—computer interfaces in several applications, including motor and speech neural prostheses. Higher-spatial-resolution micro-electrocorticography (μECoG) therefore represents a promising combination of minimal invasiveness and improved signal quality. Therefore, it would be highly beneficial for neural devices to make use of non-penetrating cortical interfaces.

### SUMMARY

The present disclosure is directed to systems and methods for utilizing transfer learning techniques for developing calibration models for neural devices comprising brain-computer interfaces and related medical devices.

In one embodiment, there is provided a computer-implemented method for calibrating a neural device, the method comprising: aggregating calibration data across a user population to define a global dataset, the calibration data comprising at least one of neural data recorded from users calibrating neural devices, neural device data from users calibrating the neural devices, or external sensor data associated with the neural devices from users calibrating the neural devices; identifying similar data segments across the global dataset to define a task-independent training dataset; training a feature extraction model based on the task-independent training dataset to define a trained, task-independent feature extraction model; receiving the calibration data from a user calibrating the neural device; and calibrating a user-specific feature extraction model using the trained, task-independent feature extraction model and the calibration data.

In one embodiment, there is provided a system comprising: a neural device; and a computer system communicably coupled to the neural device, the computer system comprising: a processor, and a memory coupled to the processor, the memory storing instructions that, when executed by the processor, cause the computer system to: aggregate calibration data across a user population to define a global dataset, the calibration data comprising at least one of neural data recorded from users calibrating neural devices, neural device data from users calibrating the neural devices, or external sensor data associated with the neural devices from users calibrating the neural devices; identify similar data segments across the global dataset to define a task-independent training dataset; train a feature extraction model based on the task-independent training dataset to define a trained, task-independent feature extraction model; receive the calibration data from a user calibrating the neural device; and calibrate a user-specific feature extraction model using the trained, task-independent feature extraction model and the calibration data.

### FIGURES

FIG. 1 depicts a block diagram of a secure neural device data transfer system, in accordance with an embodiment of the present disclosure.

FIG. 2 depicts a diagram of a neural device, in accordance with an embodiment of the present disclosure.

FIG. 3 depicts a diagram of a thin-film, microelectrode array neural device and implantation method, in accordance with an embodiment of the present disclosure.

FIG. 4 depicts a diagram of a model for decoding signals from high-bandwidth neural interfaces, in accordance with an embodiment of the present disclosure.

FIG. 5 depicts a process for calibrating neural devices utilizing transfer learning techniques, in accordance with an embodiment of the present disclosure.

### DETAILED DESCRIPTION

The present disclosure is generally directed to systems and methods for automatic calibration of mathematical models used to perform neural decoding in high-bandwidth neural interfaces. The system consists of a high-density neural interface in direct contact with the cortical or deep brain surfaces along with one or more time-synced sensors recording motor, sensory, visual, or auditory feedback from the user's body or local environment. After an initial calibration phase involving the active input of the user and training of one or more neural decoding algorithms, the system uses transfer learning techniques to create user-specific neural decoding algorithms based on global datasets, thereby minimizing the amount of training for the neural decoding algorithms that needs to be performed for each individual user.

Neural Device Systems

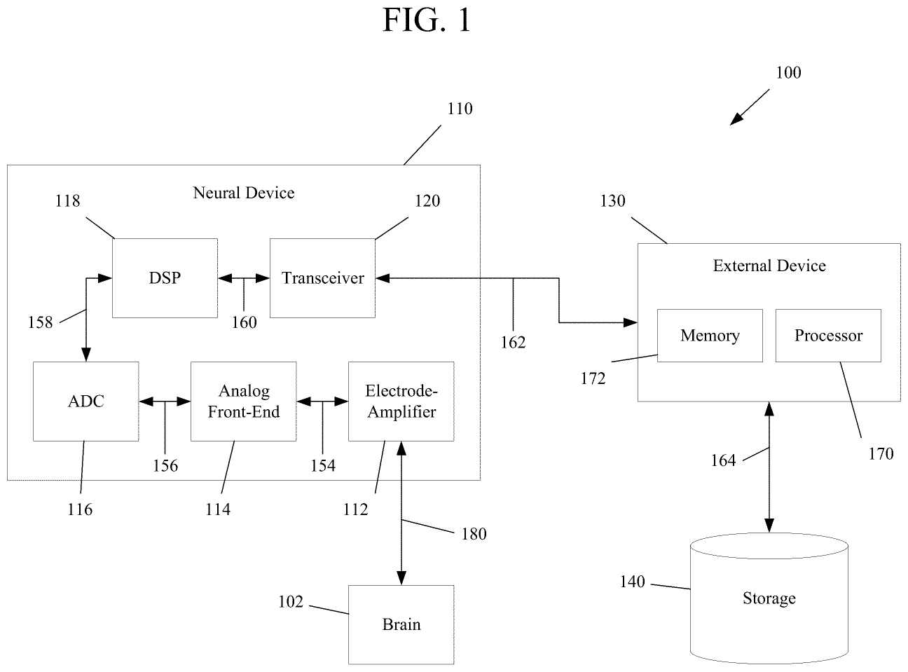

In some embodiments, the present disclosure is directed to neural devices can include electrode arrays that penetrate a subject's brain in order to sense and/or stimulate the brain. In other embodiments, the present disclosure is directed to the use of non-penetrating neural devices, i.e., neural devices having electrode arrays that do not penetrate the cortical surface. Such non-penetrating neural devices are minimally invasive and minimize the amount of impact on the subject's cortical tissue. Neural devices can sense and record brain activity, receive instructions for stimulating the subject's brain, and otherwise interact with a subject's brain as generally described herein. Referring now to FIG. 1, there is shown a diagram of an illustrative system 100 including a neural device 110 that is communicatively coupled to an external device 130. The external device 130 can include any device that the neural device 110 can be communicatively coupled, such as a computer system or mobile device (e.g., a tablet, a smartphone, a laptop, a desktop, a secure server, a smartwatch, a head-mounted virtual reality device, a head-mounted augmented reality device, or a smart inductive charger device). The external device 103 can include a processor 170 and a memory 172. In some embodiments, the computer system 102 can include a server or a cloud-based computing system. In some embodiments, the external device 130 can further include or be communicatively coupled to storage 140. In one embodiment, the storage 140 can include a database stored on the external device 130. In another embodiment, the storage 140 can include a cloud computing system (e.g., Amazon Web Services or Azure).

The neural device 110 can include a range of electrical or electronic components. In the illustrated embodiment, the neural device 110 includes an electrode-amplifier stage 112, an analog front-end stage 114, an analog-to-digital converter (ADC) stage 116, a digital signal processing (DSP) stage 118, and a transceiver stage 120 that are communicatively coupled together. The electrode-amplifier stage 112 can include an electrode array, such as is described below, that is able to physically interface with the brain of the subject 102 in order to sense brain signals and/or apply electrical signals thereto. The analog front-end stage 114 can be configured to amplify signals that are sensed from or applied to the subject 102, perform conditioning of the sensed or applied analog signals, perform analog filtering, and so on. The front-end stage 114 can include, for example, one or more application-specific integrated circuits (ASICs) or other electronics. The ADC stage 116 can be configured to convert received analog signals to digital signals. The DSP stage 118 can be configured to perform various DSP techniques, including multiplexing of digital signals received via the electrode-amplifier stage 112 and/or from the external device 130. For example, the DSP stage 118 can be configured to convert instructions from the external device 130 to a corresponding digital signal. The transceiver stage 120 can be configured to transfer data from the neural device 110 to the external device 130 located outside of the body of the subject 102.

In various embodiments, the stages of the neural device 110 can provide unidirectional or bidirectional communications (as indicated in FIG. 1) by and between the neural device 110 and the external device 130. As indicated in FIG. 1, the external device 130 and the stages 112, 114, 116, 118, 120 of the neural device 110 may be electrically coupled by connectors 154, 156, 158, 160, 162, which may be electrical wires, busses, or any type of electrical connector that enables unidirectional or bidirectional communications. In various embodiments, one or more of the stages 112, 114, 116, 118, 120 can operate in a serial or parallel manner with other stages of the system 100. It can be understood that the depicted architecture for the system 100 is merely illustrative and the system 100 can be arranged in various different manners, i.e., stages or other components of the system 100 may be connected differently and/or the system 100 may include additional or alternate stages or components. For example, any of the stages may be arranged and operate in a serial or parallel fashion with other stages of the system 100.

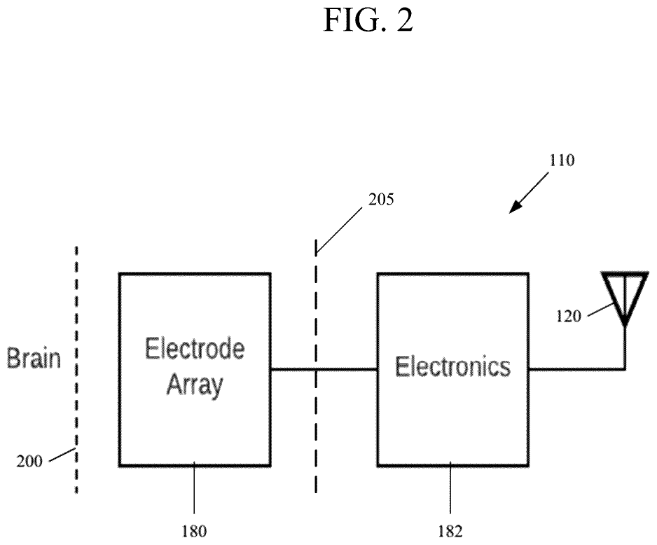

In some embodiments, the neural device 110 described above can include a brain implant, such as is shown in FIG. 2. The neural device 110 may be a biomedical device configured to study, investigate, diagnose, treat, and/or augment brain activity. In some embodiments, the neural device 110 may be a subdural neural device, i.e., a neural device implanted between the dura 205 (i.e., the membrane surrounding the brain) and the cortical surface of the brain 200. In some embodiments, the neural device 110 may be positioned beneath the dura mater 205 or between the dura mater 205 and the arachnoid membrane. In some embodiments, the neural device 110 may be positioned in the subdural space, on the cortical surface of the brain 200. The neural device 110 may be inserted through an incision in the scalp 202 and across the dura 205. The neural device 110 can include an electrode array 180 (which may be a component of or coupled to the electrode-amplifier stage 112 described above) that is configured to record and/or stimulate an area of the brain 200. The electrode array 180 can be connected to an electronics hub 182 (which can include one or more of the electrode-amplifier stage 112, analog front-end stage 114, ADC stage 116, and DSP stage 118) that is configured to transmit via wireless or wired transceiver 120 to the external device 130 (in some cases, referred to as a “receiver”).

The electrode array 180 of the neural device 110 can be of a sufficient size to measure one or more areas of interest along the cortical surface. In one embodiment, the neural device 110 can include a number of electrodes (i.e., channels) that is sufficient to measure one or more areas of the cortical surface of interest. In one embodiment, the electrode array 180 can include 500 or more electrodes. In another embodiment, the electrode array 180 can include 1,000 or more electrodes. In one illustrative embodiment, the electrode array 180 can include 1,024 electrodes.

In some embodiments, the neural device 110 can be configured to sample each channel between from about 500 Hz to about 40 kHz. In one illustrative embodiment, the neural device 110 can be configured to record electrocortical measurements at up to about 20 kHz. In another illustrative embodiment, the neural device 110 can be configured to record electrocortical measurements at up to about 30 kHz.

The electrode array 180 can comprise nonpenetrating cortical surface microelectrodes (i.e., the electrode array 180 does not penetrate the brain 200). Accordingly, the neural device 110 can provide a high spatial resolution, with minimal invasiveness and improved signal quality. The minimal invasiveness of the electrode array 180 is beneficial because it allows the neural device 110 to be used with a larger population of patients than conventional brain implants, thereby expanding the application of the neural device 110 and allowing more individuals to benefit from brain—computer interface technologies. Furthermore, the surgical procedures for implanting the neural devices 110 are minimally invasive, reversible, and avoid damaging neural tissue. In some embodiments, the electrode array 180 can be a high-density microelectrode array that provides smaller features and improved spatial resolution relative to conventional neural implants.

In some embodiments, the neural device 110 includes an electrode array configured to stimulate or record from neural tissue adjacent to the electrode array, and an integrated circuit in electrical communication with the electrode array, the integrated circuit having an analog-to-digital converter (ADC) producing digitized electrical signal output. In some embodiments, the ADC or other electronic components of the neural device 110 can include an encryption module, such as is described below. The neural device 110 can also include a wireless transmitter (e.g., the transceiver 120) communicatively coupled to the integrated circuit or the encryption module and an external device 130. The neural device 110 can also include, for example, control logic for operating the integrated circuit or electrode array 180, memory for storing recordings from the electrode array, and a power management unit for providing power to the integrated circuit or electrode array 180.

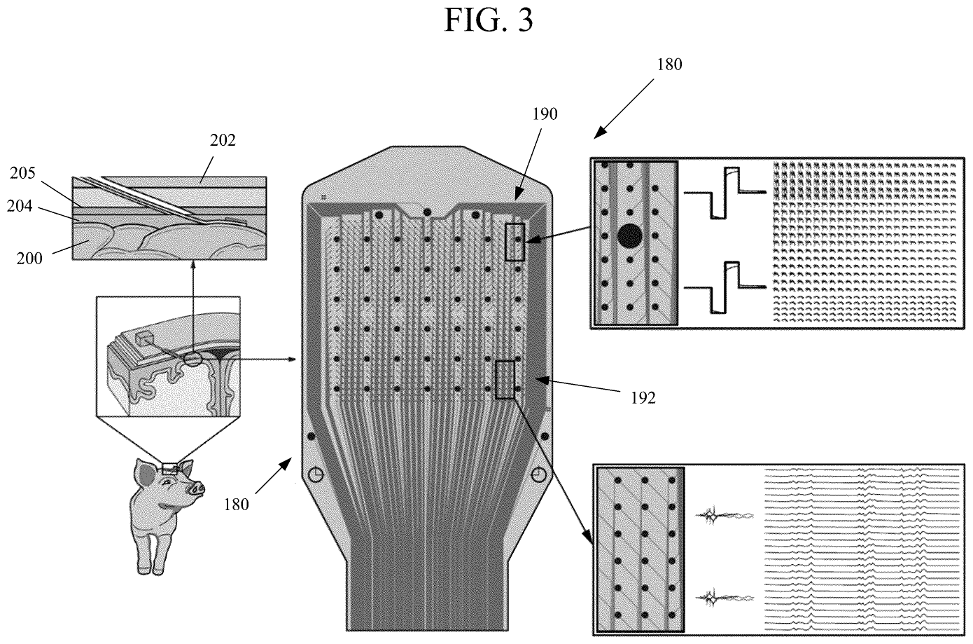

Referring now to FIG. 3, there is shown a diagram of an illustrative embodiment of a neural device 110. In this embodiment, the neural device 110 comprises an electrode array 180 comprising nonpenetrating microelectrodes. As generally described above, the neural device 110 is configured for minimally invasive subdural implantation using a cranial micro-slit technique, i.e., is inserted into the subdural space 204 between the dura 205 and the surface of the subject's brain 200. In some embodiments, the neural device 110 is inserted into the subdural space 204 between the dura 205 and the surface of the brain 200. The microelectrodes of the electrode array 180 may be arranged in a variety of different configurations and may vary in size. In some embodiments, the electrodes of the electrode array 180 can be from about 10 μm to about 500 μm in width. In one illustrative embodiment, the electrodes of the electrode array 180 can be about 50 μm in width. In some embodiments, the electrodes of the electrode array 180 can be spaced by about 200 μm (i.e., 0.2 mm) to about 3,000 μm (i.e., 3 mm). In illustrative one embodiment, adjacent electrodes of the electrode array 180 can be spaced by about 400 μm. In various embodiments the electrode array 180 can include electrodes of the same or different sizes. In this particular example, the electrode array 180 includes a first group 190 of electrodes having a first size and a second group 192 of electrodes having a second size. For example, the electrode array 180 can include recording electrodes having a particular size (e.g., 50 μm) and stimulating electrodes having a different size (e.g., 380 μm). Further, example stimulation waveforms in connection with the first group 190 of electrodes and the resulting post-stimulus activity recorded over the entire array is depicted for illustrative purposes. Still further, example traces from recorded neural activity recorded by the second group 192 of electrodes are likewise illustrated. In this example, the electrode array 180 provides multichannel data that can be used in a variety of electrophysiologic paradigms to perform neural recording of both spontaneous and stimulus-evoked neural activity as well as decoding and focal stimulation of neural activity across a variety of functional brain regions.

Additional information regarding brain-computer interfaces described herein can be found in Ho et al., The Layer 7 Cortical Interface: A Scalable and Minimally Invasive Brain—Computer Interface Platform, bioRxiv 2022.01.02.474656; doi:

https://doi.org/10.1101/2022.01.02.474656, which is hereby incorporated by reference herein in its entirety.

Transfer Learning for Neural Decoding

As generally noted above, one issue facing neural device systems, such as the system 100 described above in connection with FIG. 1, is that unique decoding algorithms must be trained for each individual patient, which is both data inefficient and limits algorithm performance. Therefore, it would be beneficial to implement transfer learning techniques in neural systems so that the decoding algorithms can be trained using less active training data from each individual patient. Further, the application of transfer learning techniques to neural decoding applications would provide the ability to build neural decoding algorithms that are more robust to real-world variation. Still further, transfer learning techniques would also be beneficial for the ability to train decoding algorithms that can perform tasks that would be infeasible to train using only a single individual's training data.

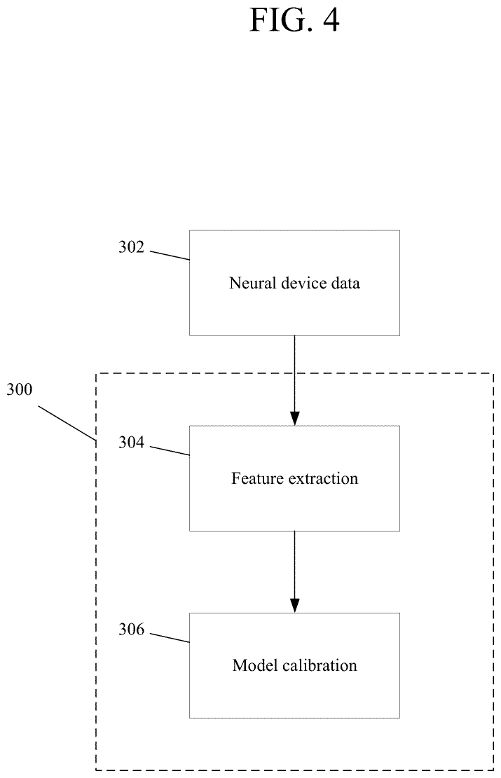

Decoding signals from high-bandwidth neural interfaces, such as described above, can conceptually be represented by a two-stage model 300, as shown in FIG. 4. The first or feature extraction stage 304 maps raw neural device data 302 to abstract features that are relevant to determining both whether and what type of stimulus or intent has occurred. The second or model calibration stage 306 calibrates the algorithm or model by mapping the features determined from the feature extraction stage 304 into a final model output. For this second stage 306, a unique decoding algorithm must be trained for each individual user. Once the neural device 110 has been implanted, the user is asked to perform a series of task-specific actions to train an initial decoding algorithm, thereby calibrating the decoding algorithm to the individual user. For example, if the neural device 110 is being used for motor decoding, the user may be asked to perform (or, if unable to do so based on disability/injury, to imagine performing) various motor activities, such as walking, moving their arm to various positions, typing or writing letters, or jumping. If the neural device 110 is being used for speech decoding applications, the user may be asked to speak (or to imagine speaking) words or sentences from a pre-defined vocabulary. While the user is performing (or imaging performing) these tasks, time-synced neural data and/or derived data is recorded from the neural device(s) 110. This neural and/or derived data can then be utilized to calibrate the decoding algorithm for the individual user. Further, the neural and/or derived data can be transmitted and/or stored by the system 100 for subsequent analysis.

As can be envisioned, the system 100 is able to obtain a large amount from each individual user as a result of the decoding algorithm calibration process for the neural device 110. Further, this data can be pooled across many different users, thereby allowing the system 100 to aggregate large amounts of data across user populations. Additionally, data collected from such high-bandwidth neural devices 110 has a high degree of dimensionality. Further, as described in U.S. Provisional patent application Ser. No. 18/491,351, titled SELF-CALIBRATING NEURAL DECODING, filed Oct. 20, 2023, which is hereby incorporated by reference herein in its entirety, the system 100 can be configured to obtain additional data that can be used to supplement the neural device data obtained during the calibration stage, including user position data obtained via external sensors, which can be further included in the transfer learning dataset. The combination of the large amounts of data and the high degree of dimensionality of the data allows the system 100 to build a global training dataset and implement transfer learning techniques for calibrating individual users' neural decoding algorithms.

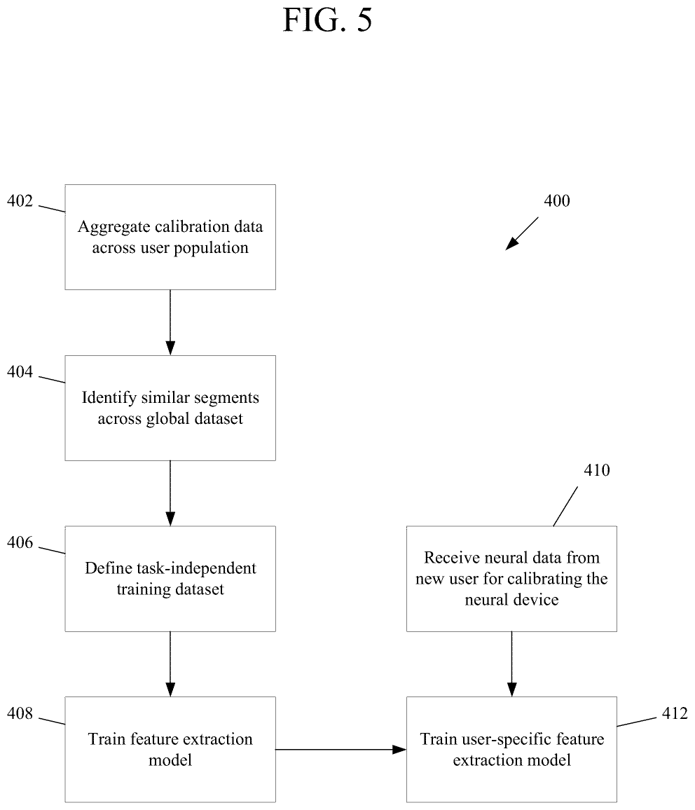

One embodiment of a process 400 for using transfer learning for calibrating neural decoding algorithms is shown in FIG. 5. In one embodiment, the process 400 can be embodied as instructions stored in a memory (e.g., the memory 172) that, when executed by a processor (e.g., the processor 170), causes the external device 130 to perform the process 400. In various embodiments, the process 400 can be embodied as software, hardware, firmware, and various combinations thereof. In various embodiments, the process 400 can be executed by and/or between a variety of different devices or systems. For example, various combinations of steps of the process 400 can be executed by the external device 130, the neural device 110, and/or other components of the system 100. In various embodiments, the system 100 executing the process 400 can utilize distributed processing, parallel processing, cloud processing, and/or edge computing techniques. The process 400 is described below as being executed by the system 100; accordingly, it should be understood that the functions can be individually or collectively executed by one or multiple devices or systems.

Turning now to the particular implantation of the process 400, a computer system (e.g., the external device 130) aggregates 402 calibration data (i.e., the data generated during model calibration 306 as discussed above) across the population of users for the neural devices 110. The data can be aggregated and/or stored in the storage 140 described above as users perform the steps of calibrating the neural devices 110. The aggregated user population data thus defines a global dataset that can be used for transfer learning techniques, as described below. In one embodiment, the aggregated 402 data can include the anatomic and functional location of the electrode array 180 from which the data was generated. In other words, the data can be labeled with the anatomic and/or functional location from which the data was generated.

Accordingly, the computer system identifies 404 similar segments across the global dataset and based thereon, defines 406 a task-independent training dataset. The similar data segments can be identified 404 based on task-based data, external sensor-based data, or other data generated from the execution of the model calibration 306 stage. In various embodiments, the segments can be identified 404 in a variety of different manners. For example, data segments can be identified 404 according to data generated from replicates of the same training task performed by an individual (e.g., typing the same letter or speaking the same word) during model training, i.e., the computer system can identify data generated from the performance on the same tasks across the user population. As another example, data segments can be identified 404 according to data generated during periods of time in which the user is not performing a decoding-relevant task. The data recorded from when users are not performing a decoding-relevant tasks can be utilized as negative or control segments for the purposes of the decoding algorithm. As yet another example, data segments can be identified 404 by performing small translational perturbations of the electrode array input. Due to the highly correlated nature of the signals in such high-resolution interfaces as used in the neural devices 110, they can be expected to produce highly similar outputs under similar calibration conditions. As yet another example, data from analogous anatomic and/or functional locations of the brain can be identified 404 across the global dataset. Regardless of the particular techniques for identifying 404 the similar segments, the computer system can enumerate sets of self-similar neural time-series data segments. Each self-similar set of data segments can be combined with all other sets of self-similar segments from other tasks to define 406 a global dataset that is independent of the specific subtask. It should further be noted that the training dataset grows combinatorically with the number of users, which can help improve the data efficiency of the process 400.

Accordingly, the computer system trains 408 a feature extraction model using the task-independent training dataset that has been created from the data aggregated across the user population. In one embodiment, the computer system can train 408 the feature extraction model based on data from an analogous anatomic and/or functional location of the brain, as noted above. In some embodiment, the feature extraction model can be trained 408 using, for example, contrastive pre-training. To gain intuition for this step, the underlying principle is that different segments of raw neural data that correspond to identical or nearly identical actions should be mapped to identical or nearly identical feature vectors in the corresponding feature space, whereas segments of neural data that correspond to segments from different actions should be mapped far away from each other in the corresponding feature space. Contrastive pre-training is one method for enforcing this restriction, by penalizing feature extraction models that map identical or nearly identical raw neural-data input pairs to very different feature vectors in the feature space and rewarding those models that map such input pairs to very similar feature vectors. However, other embodiments may use techniques other than contrastive pre-training. The particular training technique for the feature extraction model would depend on the specific nature of the decoding task.

Once the feature extraction model has been trained, it can then be used to train individual users' calibration models. In particular, a computer system and/or the neural device 110 can receive 410 neural data from a subsequent user for calibrating the decoding algorithm for the neural device 110 and train 412 the decoding algorithm using the feature extraction model that has been trained on the task-independent global dataset.

It should be noted that task-related information is only used in the process 400 in the construction of the self-similar datasets. Once the self-similar datasets have been identified 404, the feature extraction model is trained in a way that does not need to know which task was being performed at each input segment. This task-independence of the training is what allows us to pool data from different patients to create training datasets that improve overall decoding algorithm performance for one patient, using data collected by many patients. Furthermore, the computer system does not need to know what task the user is performing in order to create self-similar pairs of training data. Accordingly, external sensors (e.g., wearable or ambient monitors) can be utilized to collect training data from naturalistic behavior exhibited by the user, in addition to active training of the calibration model as described above.

Existing solutions to neural decoding have used both traditional machine learning techniques as well as various deep learning techniques; however, existing techniques have largely been trained on an individual basis, i.e., decoding algorithms for each patient are treated separately. There are many reasons for this, including the facts that (i) the majority of these systems have either been trained on single or small numbers of patients in research-use-only, academic settings, and (ii) most existing brain-computer interface decoding solutions are based on penetrating electrode technology that leverage neural spikes and/or multi-unit potential, which are inherently more unique to the individual patient and electrode location than local field potentials. Given these difficulties, the benefits of pooling data across patients to improve algorithm performance have not yet exceeded the additional cost/complexity of these solutions. However, because the neural devices 110 described herein utilize non-penetrating electrodes, the calibration of non-penetrating neural devices 110 can make use of data aggregated across patient populations in ways that neural devices that rely on penetrating electrodes are simply incapable of performing.

It should further be noted that, although the functions and/or steps of the process 400 are depicted in a particular order or arrangement, the depicted order and/or arrangement of steps and/or functions is simply provided for illustrative purposes. Unless explicitly described herein to the contrary, the various steps and/or functions of the process 400 can be performed in different orders, in parallel with each other, in an interleaved manner, and so on.

This disclosure is not limited to the particular systems, devices and methods described, as these may vary. The terminology used in the description is for the purpose of describing the particular versions or embodiments only and is not intended to limit the scope of the disclosure.

The following terms shall have, for the purposes of this application, the respective meanings set forth below. Unless otherwise defined, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention.

As used herein, the term “implantable medical device” includes any device that is at least partially introduced, either surgically or medically, into the body of a subject and is intended to remain there after the procedure.

As used herein, the singular forms “a,” “an,” and “the” include plural references, unless the context clearly dictates otherwise. Thus, for example, reference to a “protein” is a reference to one or more proteins and equivalents thereof known to those skilled in the art, and so forth.

As used herein, the term “about” means plus or minus 10% of the numerical value of the number with which it is being used. Therefore, about 50 mm means in the range of 45 mm to 55 mm.

As used herein, the term “consists of” or “consisting of” means that the device or method includes only the elements, steps, or ingredients specifically recited in the particular claimed embodiment or claim.

In embodiments or claims where the term “comprising” is used as the transition phrase, such embodiments can also be envisioned with replacement of the term “comprising” with the terms “consisting of” or “consisting essentially of.”

As used herein, the term “subject” as used herein includes, but is not limited to, humans and non-human vertebrates such as wild, domestic, and farm animals.

While the present disclosure has been illustrated by the description of exemplary embodiments thereof, and while the embodiments have been described in certain detail, it is not the intention of the Applicants to restrict or in any way limit the scope of the appended claims to such detail. Additional advantages and modifications will readily appear to those skilled in the art. Therefore, the disclosure in its broader aspects is not limited to any of the specific details, representative devices and methods, and/or illustrative examples shown and described. Accordingly, departures may be made from such details without departing from the spirit or scope of the Applicant's general inventive concept.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

In addition, even if a specific number is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, sample embodiments, or drawings, should be understood to contemplate the possibilities of including one of the terms, either of the terms, or both terms. For example, the phrase “A or B” will be understood to include the possibilities of “A” or “B” or “A and B.”

In addition, where features of the disclosure are described in terms of Markush groups, those skilled in the art will recognize that the disclosure is also thereby described in terms of any individual member or subgroup of members of the Markush group.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

1. A computer-implemented method for calibrating a neural device, the method comprising:
aggregating calibration data across a user population to define a global dataset, the calibration data comprising at least one of neural data recorded from users calibrating neural devices, neural device data from users calibrating the neural devices, or external sensor data associated with the neural devices from users calibrating the neural devices;
identifying similar data segments across the global dataset to define a task-independent training dataset;
training a feature extraction model based on the task-independent training dataset to define a trained, task-independent feature extraction model;
receiving the calibration data from a user calibrating the neural device; and
calibrating a user-specific feature extraction model using the trained, task-independent feature extraction model and the calibration data.

2. The method of claim 1, wherein the neural device comprises an electrode array comprising 1,000 or more electrodes.

3. The method of claim 1, wherein training the feature extraction model comprises contrastive pre-training.

4. The method of claim 1, wherein identifying similar data segments across the global dataset comprises utilizing data from external sensors configured to sense a characteristic or an action associated with a user.

5. The method of claim 4, wherein the external sensors comprise at least one of an inertial sensor, a camera, a tactile sensor, and a microphone.

6. The method of claim 1, further comprises:
receiving data from the neural device;
decoding the received data using the user-specific feature extraction model to define decoded data; and
determining a task associated with the decoded data.

7. The method of claim 6, wherein the task is selected from the group consisting of a motor decoding task, an auditory decoding task, a sensory decoding task, and a visual decoding task.

8. The method of claim 1, wherein identifying the similar data segments comprises:
identifying segments of the calibration data generated from replicates of a same training task performed by a plurality of individuals.

9. The method of claim 1, wherein identifying the similar data segments comprises:
identifying segments of the calibration data generated during periods of time in which the users are not performing a decoding-relevant task.

10. The method of claim 1, wherein identifying the similar data segments comprises:
performing small translational perturbations of an electrode array input of the neural devices.

11. A system comprising:
a neural device; and
a computer system communicably coupled to the neural device, the computer system comprising:
a processor, and
a memory coupled to the processor, the memory storing instructions that, when executed by the processor, cause the computer system to:
aggregate calibration data across a user population to define a global dataset, the calibration data comprising at least one of neural data recorded from users calibrating neural devices, neural device data from users calibrating the neural devices, or external sensor data associated with the neural devices from users calibrating the neural devices;
identify similar data segments across the global dataset to define a task-independent training dataset;
train a feature extraction model based on the task-independent training dataset to define a trained, task-independent feature extraction model;
receive the calibration data from a user calibrating the neural device; and
calibrate a user-specific feature extraction model using the trained, task-independent feature extraction model and the calibration data.

12. The system of claim 11, wherein the neural device comprises an electrode comprising 1,000 or more electrodes.

13. The system of claim 11, wherein the memory stores instructions that, when executed by the processor, cause the computer system to train the feature extraction model comprises contrastive pre-training.

14. The system of claim 11, further comprising:
external sensors configured to detect a characteristic or an action associated with a user;
wherein the memory stores instructions that, when executed by the processor, cause the computer system to identify similar data segments across the global dataset comprises utilizing data from the external sensors.

15. The system of claim 14, wherein the external sensors comprise at least one of an inertial sensor, a camera, a tactile sensor, and a microphone.

16. The system of claim 11, wherein the memory stores instructions that, when executed by the processor, cause the computer system to:
receive data from the neural device;
decode the received data using the user-specific feature extraction model to define decoded data; and
determine a task associated with the decoded data.

17. The system of claim 16, wherein the task is selected from the group consisting of a motor decoding task, an auditory decoding task, a sensory decoding task, and a visual decoding task.

18. The system of claim 11, wherein the memory stores instructions that, when executed by the processor, cause the computer system to identify the similar data segments by:
identifying segments of the calibration data generated from replicates of a same training task performed by a plurality of individuals.

19. The system of claim 11, wherein the memory stores instructions that, when executed by the processor, cause the computer system to identify the similar data segments by:
identifying segments of the calibration data generated during periods of time in which the users are not performing a decoding-relevant task.

20. The system of claim 11, wherein the memory stores instructions that, when executed by the processor, cause the computer system to identify the similar data segments by:
performing small translational perturbations of an electrode array input of the neural devices.
